(19) United States
(12) Patent Application Publication
(10) Pub. No.: US 2025/0144390 A1
(43) Pub. Date: May 8, 2025

(54) MEDICAL DEVICE FOR ULTRASOUND-ASSISTED DRUG DELIVERY

(71) Applicant: Boston Scientific Scimed, Inc., Maple Grove, MN (US)

(72) Inventors: Benjamin Montag, Delano, MN (US); Curtis Cornell Genstler, Snohomish, WA (US); Noah Chan Ridge, St. Paul, MN (US); Zachary Balsiger, St. Paul, MN (US); Vidhya Ramaswamy, Plymouth, MN (US)

(73) Assignee: Boston Scientific Scimed, Inc., Maple Grove, MN (US)

(21) Appl. No.: 18/933,501

(22) Filed: Oct. 31, 2024

Related U.S. Application Data
(60) Provisional application No. 63/547,201, filed on Nov. 3, 2023.

(51) Int. Cl. A61M 37/00

(52) U.S. Cl. CPC A61M 37/0092; A61M 2202/0007; A61M 2202/0468; A61M 2205/10; A61M 2205/33; A61M 2205/50

(57) ABSTRACT

Systems and methods for treating a vascular region are disclosed. An example system for treating a vascular region may include an elongate catheter shaft having a distal end region. A central lumen may be formed in the elongate catheter shaft. A treatment core may be disposed within the central lumen. The treatment core may include a plurality of ultrasound transducers. A control unit may be coupled to the treatment core. The control unit may be configured to shift between in a first configuration. A fluid delivery lumen may be defined in the elongate catheter shaft. A pump may be coupled to the elongate catheter shaft. The pump may be configured to deliver microbubbles, nanodroplets, or both to the fluid delivery lumen when the control unit is in the inactive state.

### CROSS-REFERENCE TO RELATED APPLICATIONS

This application claims the benefit of priority under 35 U.S.C. § 119 of U.S. Provisional Application No. 63/547,201, filed Nov. 3, 2023, the entire disclosure of which is hereby incorporated by reference.

### TECHNICAL FIELD

The present disclosure pertains to medical devices, and methods for manufacturing medical devices. More particularly, the present disclosure pertains to medical devices for ultrasound-assisted drug delivery.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A system for treating a vascular region is disclosed. The system comprises: an elongate catheter shaft having a distal end region; wherein a central lumen is formed in the elongate catheter shaft; a treatment core disposed within the central lumen, the treatment core including a plurality of ultrasound transducers disposed adjacent to the distal end region of the elongate catheter shaft; a control unit coupled to the treatment core, the control unit being configured to shift between in a first configuration where the plurality of ultrasound transducers are in an active state and a second configuration where the plurality of ultrasound transducers are in an inactive state; a fluid delivery lumen defined in the elongate catheter shaft; and a pump coupled to the elongate catheter shaft, the pump being configured to deliver microbubbles, nanodroplets, or both to the fluid delivery lumen when the ultrasound transducers are in the inactive state.

Alternatively or additionally to any of the embodiments above, the pump is configured to deliver saline through the fluid delivery lumen when the when the ultrasound transducers are in the active state.

Alternatively or additionally to any of the embodiments above, the pump includes a switch.

Alternatively or additionally to any of the embodiments above, the switch is configured to switch between a first switch configuration where the pump delivers microbubbles, nanodroplets, or both to the fluid delivery lumen and a second switch configuration where the pump delivers saline to the fluid delivery lumen.

Alternatively or additionally to any of the embodiments above, when the control unit is in the first configuration, the plurality of ultrasound transducers are in the active state for about 10-60 seconds.

Alternatively or additionally to any of the embodiments above, when the control unit is in the first configuration, the plurality of ultrasound transducers are in the active state for about 15-45 seconds.

Alternatively or additionally to any of the embodiments above, when the control unit is in the first configuration, the plurality of ultrasound transducers are in the active state for about 30 seconds.

Alternatively or additionally to any of the embodiments above, when the control unit is in the second configuration, the plurality of ultrasound transducers are in the inactive state for about 10-60 seconds.

Alternatively or additionally to any of the embodiments above, when the control unit is in the second configuration, the plurality of ultrasound transducers are in the inactive state for about 15-45 seconds.

Alternatively or additionally to any of the embodiments above, when the control unit is in the second configuration, the plurality of ultrasound transducers are in the inactive state for about 30 seconds.

Alternatively or additionally to any of the embodiments above, the pump is configured to not deliver microbubbles, nanodroplets, or both to the fluid delivery lumen when the ultrasound transducers are in the active state.

A system for treating a vascular region is disclosed. The system comprises: an elongate catheter shaft; a treatment core disposed within the elongate catheter shaft, the treatment core including a plurality of ultrasound transducers; a control unit coupled to the treatment core, the control unit being configured to shift between in a first state where the plurality of ultrasound transducers are active and a second state where the plurality of ultrasound transducers are inactive; a fluid delivery lumen defined in the elongate catheter shaft; and a pump coupled to the elongate catheter shaft, wherein the pump is configured to deliver microbubbles, nanodroplets, or both to the fluid delivery lumen when the control unit is in the first state and wherein the pump is configured to not deliver microbubbles, nanodroplets, or both to the fluid delivery lumen when the control unit is in the second state.

Alternatively or additionally to any of the embodiments above, the pump is configured to deliver saline through the fluid delivery lumen when the control unit is in the second state.

Alternatively or additionally to any of the embodiments above, the pump includes a switch.

Alternatively or additionally to any of the embodiments above, the switch is configured to switch between a first switch configuration when the control unit is in the first state so that the pump delivers microbubbles, nanodroplets, or both to the fluid delivery lumen and a second switch configuration when the control unit is in the second state so that the pump delivers saline to the fluid delivery lumen.

Alternatively or additionally to any of the embodiments above, when the control unit is in the first state, the plurality of ultrasound transducers are active for about 10-60 seconds.

Alternatively or additionally to any of the embodiments above, when the control unit is in the first state, the plurality of ultrasound transducers are active for about 15-45 seconds.

Alternatively or additionally to any of the embodiments above, when the control unit is in the first state, the plurality of ultrasound transducers are active for about 30 seconds.

Alternatively or additionally to any of the embodiments above, when the control unit is in the second state, the plurality of ultrasound transducers are inactive for about 10-60 seconds.

A method for delivering a drug to a vascular region is disclosed. The method comprises: advancing a catheter system to a treatment site, the catheter system comprising: an elongate catheter shaft having a distal end region, wherein a central lumen is formed in the elongate catheter shaft, a treatment core disposed within the central lumen, the treatment core including a plurality of ultrasound transducers disposed adjacent to the distal end region of the elongate catheter shaft, a control unit coupled to the treatment core, the control unit being configured to shift between in a first configuration where the plurality of ultrasound transducers are in an active state and a second configuration where the plurality of ultrasound transducers are in an inactive state, a fluid delivery lumen defined in the elongate catheter shaft, and a pump coupled to the elongate catheter shaft, the pump being configured to deliver microbubbles, nanodroplets, or both to the fluid delivery lumen when the control unit is in the inactive state; advancing the treatment core through the central lumen so that the plurality of ultrasound transducers are disposed adjacent to the distal end region of the elongate catheter shaft; delivering microbubbles, nanodroplets, or both to the fluid delivery lumen when the control unit is in the inactive state; and activating at least some of the plurality of ultrasound transducers.

A system for treating a vascular region is disclosed. The system comprises: an elongate catheter shaft having a distal end region which includes a plurality of ultrasound transducers; a control unit coupled to the ultrasound transducers, the control unit being configured to shift between in a first configuration where the plurality of ultrasound transducers are in an active state and a second configuration where the plurality of ultrasound transducers are in an inactive state; a fluid delivery lumen defined in the elongate catheter shaft; and a pump coupled to the elongate catheter shaft, the pump being configured to deliver microbubbles, nanodroplets, or both to the fluid delivery lumen when the ultrasound transducers are in the inactive state.

Alternatively or additionally to any of the embodiments above, the pump is configured to deliver saline or another solution through the fluid delivery lumen when the ultrasound transducers are in the active state.

Alternatively or additionally to any of the embodiments above, the pump includes a switch.

Alternatively or additionally to any of the embodiments above, the switch is configured to switch between a first switch configuration where the pump delivers microbubbles, nanodroplets, or both to the fluid delivery lumen and a second switch configuration where the pump delivers saline or another solution to the fluid delivery lumen.

Alternatively or additionally to any of the embodiments above, when the control unit is in the first configuration, the plurality of ultrasound transducers are in the active state for about 10-60 seconds.

Alternatively or additionally to any of the embodiments above, when the control unit is in the first configuration, the plurality of ultrasound transducers are in the active state for about 15-45 seconds.

Alternatively or additionally to any of the embodiments above, when the control unit is in the first configuration, the plurality of ultrasound transducers are in the active state for about 30 seconds.

Alternatively or additionally to any of the embodiments above, when the control unit is in the second configuration, the plurality of ultrasound transducers are in the inactive state for about 10-60 seconds.

Alternatively or additionally to any of the embodiments above, when the control unit is in the second configuration, the plurality of ultrasound transducers are in the inactive state for about 15-45 seconds.

Alternatively or additionally to any of the embodiments above, when the control unit is in the second configuration, the plurality of ultrasound transducers are in the inactive state for about 30 seconds.

Alternatively or additionally to any of the embodiments above, the pump is configured to not deliver microbubbles, nanodroplets, or both to the fluid delivery lumen when the ultrasound transducers are in the active state.

A system for treating a vascular region is disclosed. The system comprises: an elongate catheter shaft having a distal end region which includes a plurality of ultrasound transducers; a control unit coupled to the ultrasound transducers, the control unit being configured to shift between in a first state where the plurality of ultrasound transducers are active and a second state where the plurality of ultrasound transducers are inactive; a fluid delivery lumen defined in the elongate catheter shaft; and a pump coupled to the elongate catheter shaft, wherein the pump is configured to deliver microbubbles, nanodroplets, or both to the fluid delivery lumen when the ultrasound transducers are in the active state.

Alternatively or additionally to any of the embodiments above, the pump is configured to deliver saline or another solution through the fluid delivery lumen when the ultrasound transducers are in the inactive state.

Alternatively or additionally to any of the embodiments above, the pump includes a switch.

Alternatively or additionally to any of the embodiments above, the switch is configured to switch between a first switch configuration where the pump delivers microbubbles, nanodroplets, or both to the fluid delivery lumen and a second switch configuration where the pump delivers saline or another solution to the fluid delivery lumen.

Alternatively or additionally to any of the embodiments above, when the control unit is in the first state, the plurality of ultrasound transducers are active for about 10-60 seconds.

Alternatively or additionally to any of the embodiments above, when the control unit is in the first state, the plurality of ultrasound transducers are active for about 15-45 seconds.

Alternatively or additionally to any of the embodiments above, when the control unit is in the first state, the plurality of ultrasound transducers are active for about 30 seconds.

Alternatively or additionally to any of the embodiments above, when the control unit is in the second state, the plurality of ultrasound transducers are inactive for about 10-60 seconds.

Alternatively or additionally to any of the embodiments above, when the control unit is in the second configuration, the plurality of ultrasound transducers are in the inactive state for about 15-45 seconds.

Alternatively or additionally to any of the embodiments above, when the control unit is in the second configuration, the plurality of ultrasound transducers are in the inactive state for about 30 seconds.

Alternatively or additionally to any of the embodiments above, the pump is configured to not deliver microbubbles, nanodroplets, or both to the fluid delivery lumen when the ultrasound transducers are in the inactive state.

A method for delivering a drug to a vascular region is disclosed. The method comprises: advancing a catheter system to a treatment site, the catheter system comprising: an elongate catheter shaft having a distal end region, wherein a central lumen is formed in the elongate catheter shaft, a treatment core disposed within the central lumen, the treatment core including a plurality of ultrasound transducers disposed adjacent to the distal end region of the elongate catheter shaft, a control unit coupled to the treatment core, the control unit being configured to shift between in a first configuration where the plurality of ultrasound transducers are in an active state and a second configuration where the plurality of ultrasound transducers are in an inactive state, a fluid delivery lumen defined in the elongate catheter shaft, and a pump coupled to the elongate catheter shaft, the pump being configured to deliver microbubbles, nanodroplets, or both to the fluid delivery lumen when the control unit is in the inactive state; advancing the treatment core through the central lumen so that the plurality of ultrasound transducers are disposed adjacent to the distal end region of the elongate catheter shaft; delivering microbubbles, nanodroplets, or both to the fluid delivery lumen when the control unit is in the inactive state; and activating at least some of the plurality of ultrasound transducers.

Alternatively or additionally to any of the embodiments above, the delivering of microbubbles, nanodroplets, or both to the fluid delivery lumen when the control unit is in the inactive state followed by activating at least some of the plurality of ultrasound transducers is repeated multiple times during the drug delivery method.

A system for treating a vascular region is disclosed. The system comprises: an elongate catheter shaft having a distal end region which includes a plurality of ultrasound transducers; a control unit coupled to the ultrasound transducers, the control unit being configured to shift between in a first configuration for a period of time where the plurality of ultrasound transducers are in an active state and a second configuration for a period of time where the plurality of ultrasound transducers are in an inactive state; a fluid delivery lumen defined in the elongate catheter shaft; and a pump coupled to the elongate catheter shaft, the pump being configured to deliver microbubbles, nanodroplets, or both to the fluid delivery lumen for a period of time that can be controlled and can vary from being in phase with the period of time the ultrasound transducers are active to being out of phase with the period of time the ultrasound transducers are active.

Alternatively or additionally to any of the embodiments above, the pump is configured to deliver saline or another solution through the fluid delivery lumen when not delivering microbubbles, nanodroplets, or both to the fluid delivery lumen.

Alternatively or additionally to any of the embodiments above, the pump includes a switch.

Alternatively or additionally to any of the embodiments above, the switch is configured to switch between a first switch configuration where the pump delivers microbubbles, nanodroplets, or both to the fluid delivery lumen and a second switch configuration where the pump delivers saline or another solution to the fluid delivery lumen.

Alternatively or additionally to any of the embodiments above, when the control unit is in the first configuration, the plurality of ultrasound transducers are in the active state for about 10-60 seconds.

Alternatively or additionally to any of the embodiments above, the control unit is in the first configuration, the plurality of ultrasound transducers are in the active state for about 15-45 seconds.

Alternatively or additionally to any of the embodiments above, when the control unit is in the first configuration, the plurality of ultrasound transducers are in the active state for about 30 seconds.

Alternatively or additionally to any of the embodiments above, when the control unit is in the second configuration, the plurality of ultrasound transducers are in the inactive state for about 10-60 seconds.

Alternatively or additionally to any of the embodiments above, when the control unit is in the second configuration, the plurality of ultrasound transducers are in the inactive state for about 15-45 seconds.

Alternatively or additionally to any of the embodiments above, when the control unit is in the second configuration, the plurality of ultrasound transducers are in the inactive state for about 30 seconds.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic illustration of certain features of an illustrative ultrasonic catheter.

FIG. 2 is a cross-sectional view taken along line 2-2 of FIG. 1.

FIG. 3 is a schematic illustration of an illustrative elongate inner core configured to be positioned within the central lumen of the catheter shown in FIG. 2.

FIG. 4 is a cross-sectional view taken along line 4-4 of FIG. 3.

FIG. 5 is a schematic wiring diagram illustrating a technique for electrically connecting five groups of ultrasound radiating members to form an ultrasound assembly.

FIG. 6 is a schematic wiring diagram illustrating a technique for electrically connecting one of the groups of FIG. 5.

FIG. 7A is a schematic illustration of the ultrasound assembly of FIG. 5 housed within the inner core of FIG. 4.

FIG. 7B is a cross-sectional view taken along line 7B-7B of FIG. 7A.

FIG. 7C is a cross-sectional view taken along line 7C-7C of FIG. 7A.

FIG. 7D is a side view of an ultrasound assembly center wire twisted into a helical configuration.

FIG. 8 illustrates the energy delivery section of the inner core of FIG. 4 positioned within the energy delivery section of the tubular body of FIG. 1.

FIG. 9 illustrates a portion of an example system.

FIG. 10 schematically illustrate a control algorithm for use with an example system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

As used herein, the term “ultrasonic energy” is used broadly, includes its ordinary meaning, and further includes mechanical energy transferred through pressure or compression waves with a frequency greater than about 20 kHz. Ultrasonic energy waves have a frequency between about 500 kHz and about 20 MHz in one example embodiment, between about 1 MHz and about 3 MHz in another example embodiment, of about 3 MHz in another example embodiment, and of about 2 MHz in another example embodiment.

As used herein, the term “catheter” is used broadly, includes its ordinary meaning, and further includes an elongate flexible tube configured to be inserted into the body of a patient, such as into a body part, cavity, duct or vessel.

As used herein, the term “therapeutic compound” is used broadly, includes its ordinary meaning, and encompasses drugs, medicaments, dissolution compounds, genetic materials, and other substances capable of effecting physiological functions. A mixture comprising such substances is encompassed within this definition of “therapeutic compound”.

As used herein, the term “end” is used broadly, includes its ordinary meaning, and further encompasses a region generally, such that “proximal end” includes “proximal region”, and “distal end” includes “distal region”.

As expounded herein, ultrasonic energy may be used to enhance the delivery and/or effect of a therapeutic compound. For example, in the context of treating vascular occlusions, ultrasonic energy has been shown to increase enzyme mediated thrombolysis by enhancing the delivery of thrombolytic agents into a thrombus, where such agents lyse the thrombus by degrading the fibrin that forms the thrombus. The thrombolytic activity of the agent is enhanced in the presence of ultrasonic energy in the thrombus. In other applications, ultrasonic energy has also been shown to enhance transfection of gene-based drugs into cells, and augment transfer of chemotherapeutic drugs into tumor cells. Ultrasonic energy delivered from within a patient's body has been found to be capable of producing non-thermal effects that increase biological tissue permeability to therapeutic compounds by up to or greater than an order of magnitude.

Use of an ultrasound catheter to deliver ultrasonic energy and a therapeutic compound directly to the treatment site mediates or overcomes many of the disadvantages associated with systemic drug delivery, such as low efficiency, high therapeutic compound use rates, and significant side effects caused by high doses. Local therapeutic compound delivery has been found to be advantageous in the context of thrombolytic therapy, chemotherapy, radiation therapy, and gene therapy, as well as in applications calling for the delivery of proteins and/or therapeutic humanized antibodies. However, it should be appreciated that in certain arrangements the ultrasound catheter can also be used in combination with systemic drug delivery instead or in addition to local drug delivery. In addition, local drug delivery can be accomplished through the use of a separate device (e.g., catheter).

As will be described below, the ultrasound catheter can include one or more ultrasound radiating members positioned therein. Such ultrasound radiating members can include a transducer (e.g., a PZT transducer), which is configured to convert electrical energy into ultrasonic energy. In such embodiments, the PZT transducer is excited by specific electrical parameters (herein “power parameters” that cause it to vibrate in a way that generates ultrasonic energy).

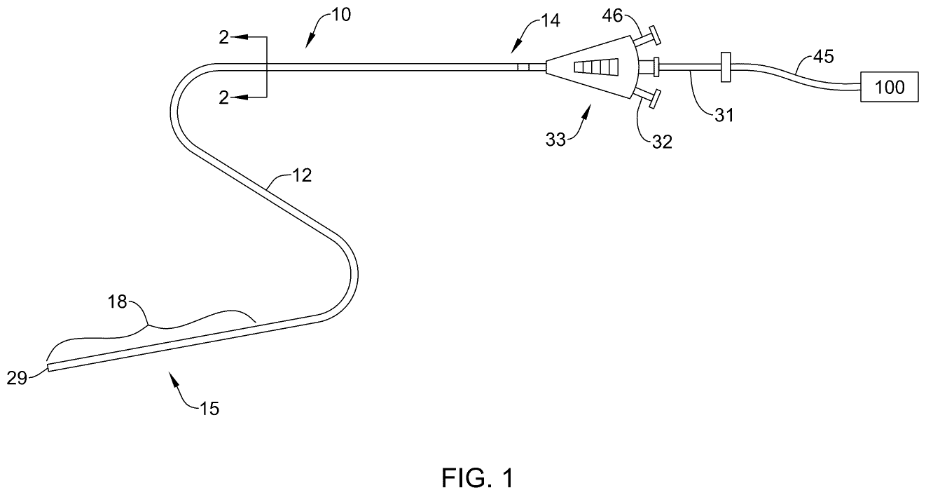

With reference to the illustrated embodiments, FIG. 1 illustrates an ultrasonic catheter 10 configured for use in a patient's vasculature. For example, in certain applications the ultrasonic catheter 10 is used to treat long segment peripheral arterial occlusions, such as those in the vascular system of the leg, while in other applications the ultrasonic catheter 10 is used to treat occlusions in the small vessels of the neurovasculature or other portions of the body (e.g., other portions of the vascular system). Thus, the dimensions of the catheter 10 may be adjusted based on the particular application for which the catheter 10 is to be used.

The ultrasonic catheter or catheter system 10 generally includes a multi-component, elongate flexible tubular body or catheter shaft 12 having a proximal end region 14 and a distal end region 15. The catheter shaft 12 includes a flexible energy delivery section 18 located in the distal end region 15 of the catheter 10. The catheter shaft 12 and other components of the catheter 10 are manufactured in accordance with a variety of techniques. Suitable materials and dimensions are selected based on the natural and anatomical dimensions of the treatment site and on the desired percutaneous access site.

For example, in an embodiment, the proximal end region 14 of the catheter shaft 12 may include a material that has sufficient flexibility, kink resistance, rigidity and structural support to push the energy delivery section 18 through the patient's vasculature to a treatment site. Examples of such materials include, but are not limited to, extruded polytetrafluoroethylene (PTFE), polyethylenes (PE), polyamides and other similar materials. In certain embodiments, the proximal end region 14 of the catheter shaft 12 may be reinforced by braiding, mesh or other constructions to provide increased kink resistance and pushability. For example, in certain embodiments nickel titanium or stainless steel wires may be placed along or incorporated into the catheter shaft 12 to reduce kinking.

In some instances, the energy delivery section 18 of the catheter shaft 12 may be formed of a material that (a) is thinner than the material forming the proximal end region 14 of the catheter shaft 12, or (b) has a greater acoustic transparency than the material forming the proximal end region 14 of the catheter shaft 12. Thinner materials generally have greater acoustic transparency than thicker materials. Suitable materials for the energy delivery section 18 include, but are not limited to, high or low density polyethylenes, urethanes, nylons, and the like. In some embodiments, the energy delivery section 18 is formed from the same material or a material of the same thickness as the proximal end region 14.

One or more fluid delivery lumens may be incorporated into the catheter shaft 12. For example, in one embodiment a central lumen passes through the catheter shaft 12. The central lumen extends through the length of the catheter shaft 12, and is coupled to a distal exit port 29 and a proximal access port 31. The proximal access port 31 forms part of a hub 33, which is attached to the proximal end region 14 of the catheter 10. In some cases, the hub 33 may include a cooling fluid fitting 46, which is hydraulically connected to a lumen within the catheter shaft 12. In some cases, the hub 33 may also include a therapeutic compound inlet port 32, which is hydraulically connected to a lumen within the catheter shaft 12. In some cases, the therapeutic compound inlet port 32 may also be hydraulically coupled to a source of therapeutic compound via a hub such as a Luer fitting.

The catheter 10 is configured to have one or more ultrasound radiating members positioned therein. For example, in certain embodiments an ultrasound radiating member may be fixed within the energy delivery section 18 of the tubular body, while in other embodiments a plurality of ultrasound radiating members are fixed to an assembly that is passed into the central lumen. In either case, the one or more ultrasound radiating members are electrically coupled to a control system 100 via a cable 45. In one embodiment, the outer surface of the energy delivery section 18 can include a cavitation promoting surface configured to enhance/promote cavitation at the treatment site. In some cases, a cavitation promoting surface is a textured surface that can retain small pockets of air when submerged. The small pockets of air can serve as a source for microbubbles or nanobubbles, thereby reducing the threshold for cavitation in an ultrasound field. In some cases, the outer surface of the energy delivery section 18 may be coated with a coating that includes components that will lower the cavitation threshold. As an example, the surface may be hydrophobic and textured in a way so that the textured surface presents a lower cavitation threshold than the surrounding bulk fluid. This can enhance the therapeutic effect of the ultrasound.

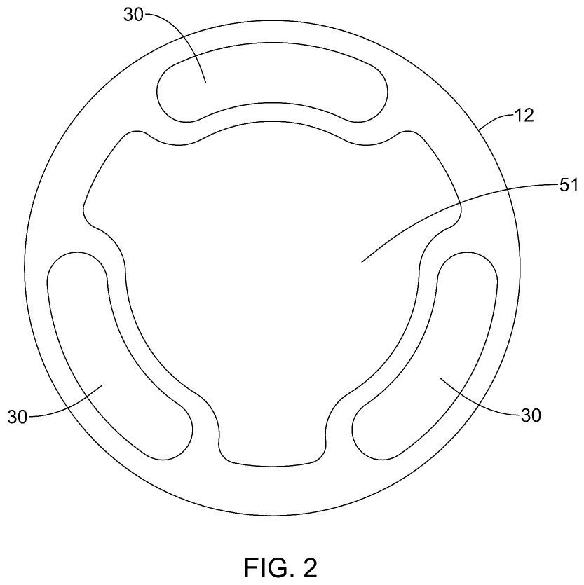

With reference to FIGS. 2-8, an exemplary arrangement of the energy delivery section 18 and other portions of the catheter 10 described above is shown. This arrangement may be well-suited for treatment of peripheral vascular occlusions.

FIG. 2 illustrates a cross section of the catheter shaft 12 taken along line 2-2 of FIG. 1. As shown in FIG. 2, three fluid delivery lumens 30 may be incorporated into the catheter shaft 12. In other embodiments, more or fewer fluid delivery lumens can be incorporated into the catheter shaft 12. The catheter shaft 12 may include a hollow central lumen 51 passing through the catheter shaft 12. The cross-section of the catheter shaft 12, as illustrated in FIG. 2, may be substantially constant along most of the length of the catheter 10. Thus, in such embodiments, substantially the same cross-section is present in both the proximal end region 14 and the distal end region 15 of the catheter 10. In some cases, the cross-section may vary within the energy delivery section 18, as will be discussed subsequently.

In certain embodiments, the central lumen 51 has a minimum diameter greater than about 0.030 inches (about 0.0762 centimeters). In another embodiment, the central lumen 51 has a minimum diameter greater than about 0.037 inches (about 0.09398 centimeters). In one example embodiment, the fluid delivery lumens 30 have dimensions of about 0.026 inches (about 0.06604 centimeters) wide by about 0.0075 inches (about 0.01905 centimeters) high, although other dimensions may be used in other applications.

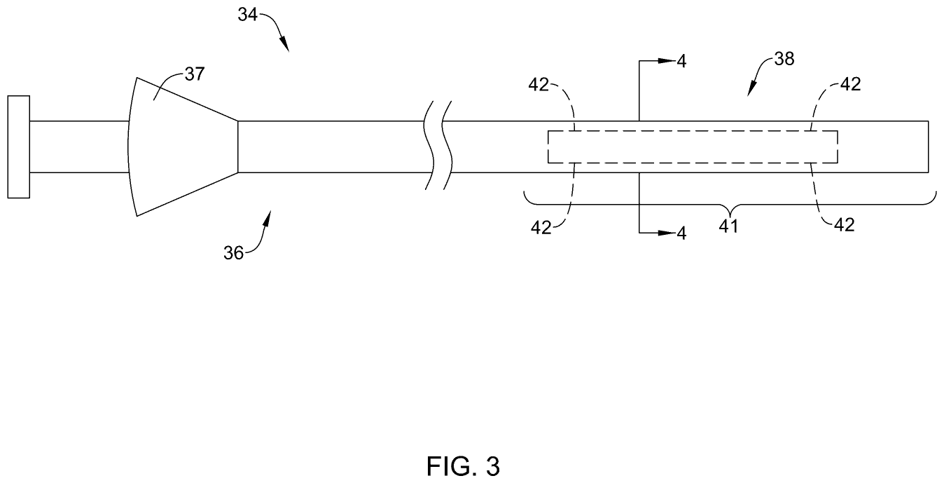

As described above, the central lumen 51 may extend through the length of the catheter shaft 12. As illustrated in FIG. 1, the central lumen 51 includes a distal exit port 29 and a proximal access port 31. The proximal access port 31 forms part of the hub 33, which is attached to the proximal end region 14 of the catheter 10. The central lumen 51 may be configured to receive an elongate inner core 34 (e.g., which may also be termed the treatment core 34) of which an embodiment is illustrated in FIG. 3. In some cases, the elongate inner core 34 includes a proximal region 36 and a distal region 38. A proximal hub 37 is fitted on the inner core 34 at one end of the proximal region 36. One or more ultrasound radiating members 40 (e.g., may also be termed ultrasound transducers 40) are positioned within an inner core energy delivery section 41 located within the distal region 38. The ultrasound radiating members 40 form an ultrasound assembly 42, which will be described in detail below.

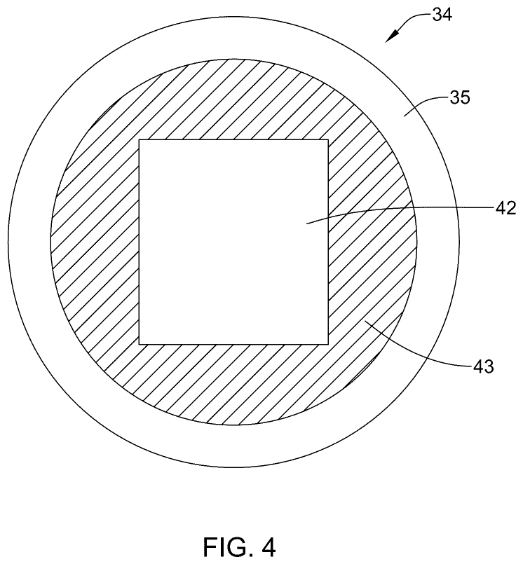

As shown in the cross-section illustrated in FIG. 4, which is taken along the line 4-4 of FIG. 3, the inner core 34 may have a cylindrical shape, with an outer diameter that permits the inner core 34 to be inserted into the central lumen 51 of the catheter shaft 12 via the proximal access port 31. Suitable outer diameters of the inner core 34 include, but are not limited to, about 0.010 inches (about 0.0254 centimeters) to about 0.100 inches (about 0.254 centimeters). In another embodiment, the outer diameter of the inner core 34 is between about 0.020 inches (about 0.0508 centimeters) and about 0.080 inches (about 0.2032 centimeters). In yet another embodiment, the inner core 34 has an outer diameter of about 0.035 inches (about 0.0889 centimeters).

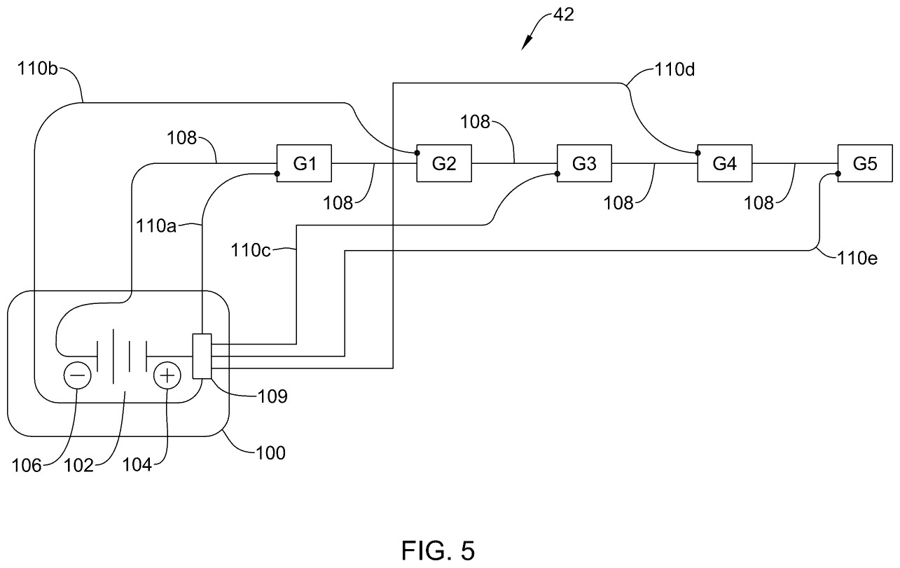

Still referring to FIG. 4, the inner core 34 may include a cylindrical outer body 35 that houses the ultrasound assembly 42. The ultrasound assembly 42 includes wiring and ultrasound radiating members, described in greater detail in FIGS. 5-7D, such that the ultrasound assembly 42 is capable of radiating ultrasonic energy from the energy delivery section 41 of the inner core 34. The ultrasound assembly 42 is electrically connected to the hub 33, where the inner core 34 can be connected to a control system 100 via cable 45 (illustrated in FIG. 1). In some cases, an electrically insulating potting material 43 fills the inner core 34, surrounding the ultrasound assembly 42, thus preventing movement of the ultrasound assembly 42 with respect to the outer body 35. In one embodiment, the thickness of the outer body 35 is between about 0.0002 inches (about 0.000508centimeters) and 0.010 inches (0.0254 centimeters). In another embodiment, the thickness of the outer body 35 is between about 0.0002 inches (about 0.000508 centimeters) and 0.005 inches (0.0127 centimeters). In yet another embodiment, the thickness of the outer body 35 is about 0.0005 inches (about 0.00127 centimeters).

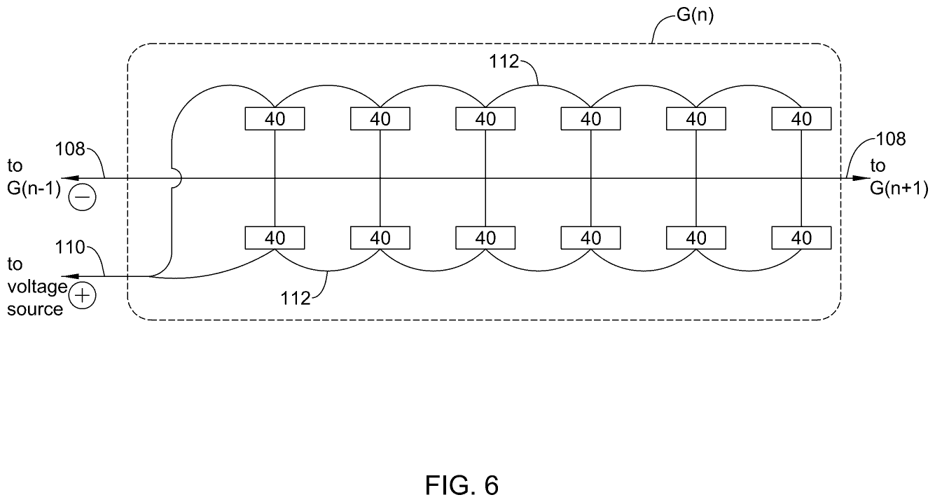

In some embodiments, the ultrasound assembly 42 includes a plurality of ultrasound radiating members 40 that are divided into one or more groups. For example, FIGS. 5-6 are schematic wiring diagrams illustrating a technique for connecting five groups of ultrasound radiating members 40 to form the ultrasound assembly 42. The ultrasound assembly 42 includes a set of transducer drivers 109, which includes a transducer driver that drives each of the five groups G1, G2, G3, G4, G5 of ultrasound radiating members 40 via electrical connections 110a, 110b, 110c, 110d and 110e, respectively. The five groups G1, G2, G3, G4, G5 of ultrasound radiating members 40 are also electrically connected to the control system 100. In some cases, a single amplifier is used, with a MUX to drive each of the individual groups, for example.

As used herein, the terms “ultrasonic energy”, “ultrasound” and “ultrasonic” are broad terms, having their ordinary meanings, and further refer to, without limitation, mechanical energy transferred through longitudinal pressure or compression waves. Ultrasonic energy can be emitted as continuous or pulsed waves, depending on the requirements of a particular application. Additionally, ultrasonic energy can be emitted in waveforms having various shapes, such as sinusoidal waves, triangle waves, square waves, or other wave forms. Ultrasonic energy includes sound waves. In certain embodiments, the ultrasonic energy has a frequency between about 20 kHz and about 20 MHz. For example, in one embodiment, the waves have a frequency between about 500 kHz and about 20 MHz. In another embodiment, the waves have a frequency between about 1 MHz and about 3 MHz. In yet another embodiment, the waves have a frequency of about 2 MHz. The average acoustic power for each ultrasound radiating member 40 is between about 0.01 watts and 300 watts. In some embodiments, the average acoustic power for each ultrasound radiating member 40 is about 0.2 watts and about 2.5 watts. In an embodiment, the average acoustic power for each ultrasound radiating member 40 is about 0.27 watts.

As used herein, the term “ultrasound radiating member” refers to any apparatus capable of producing ultrasonic energy. For example, in one embodiment, an ultrasound radiating member comprises an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics may include a crystalline material, such as quartz, that changes shape when an electrical current is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

Still referring to FIG. 5, the control system 100 may include, among other things, a voltage source 102. The voltage source 102 includes a positive terminal 104 and a negative terminal 106. The negative terminal 106 is connected to common wire 108, which connects the five groups G1-G5 of ultrasound radiating members 40 in series. The positive terminal 104 is connected to a plurality of lead wires 110a, 110b, 110c, 110d and 110e, which connect to one of the five groups G1-G5 of ultrasound radiating members 40, respectively. Thus, under this configuration, each of the five groups G1-G5, one of which is illustrated in FIG. 6, is connected to the positive terminal 104 via one of the lead wires 110a, 110b, 110c, 110d and 110e, and to the negative terminal 106 via the common wire 108. The control circuitry can be configured as part of the control system 100 and can include circuits, control routines, controllers etc. configured to vary one or more power parameters used to drive ultrasound radiating members 40.

Referring now to FIG. 6, each group G1-G5 includes a plurality of ultrasound radiating members 40. Each of the ultrasound radiating members 40 is electrically connected to the common wire 108 and to the lead wire 110 via one of two positive contact wires 112. Thus, when wired as illustrated, a constant voltage difference will be applied to each ultrasound radiating member 40 in the group. Although the group illustrated in FIG. 6 includes twelve ultrasound radiating members 40, one of ordinary skill in the art will recognize that more or fewer ultrasound radiating members 40 can be included in the group. Likewise, more or fewer than five groups can be included within the ultrasound assembly 42 illustrated in FIG. 5.

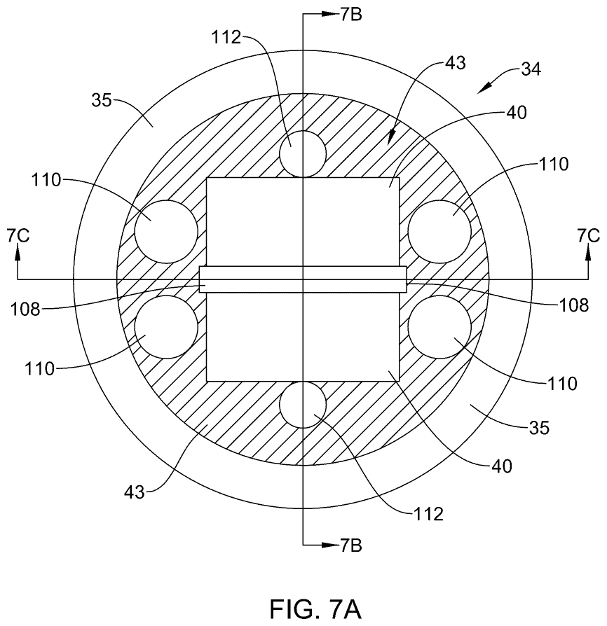

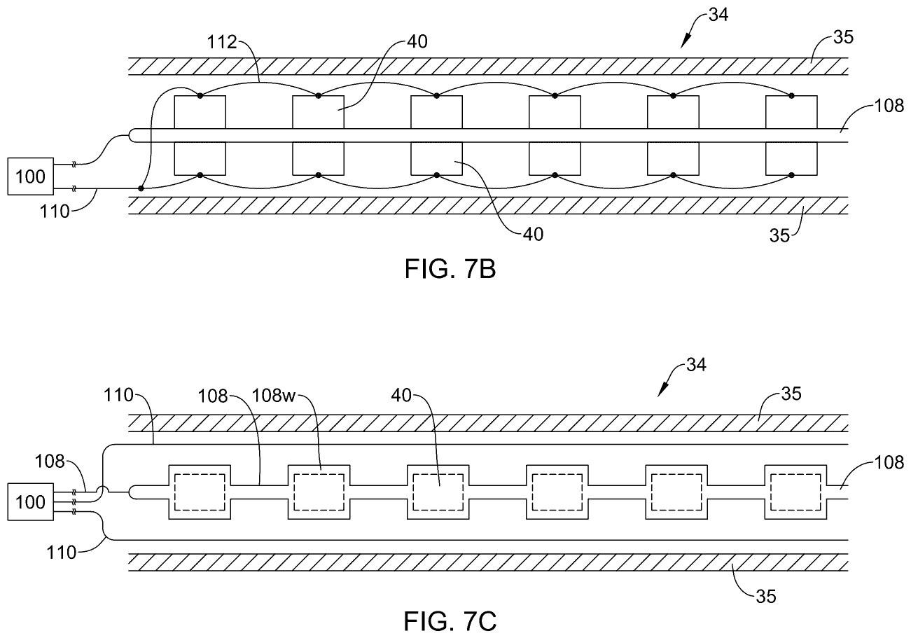

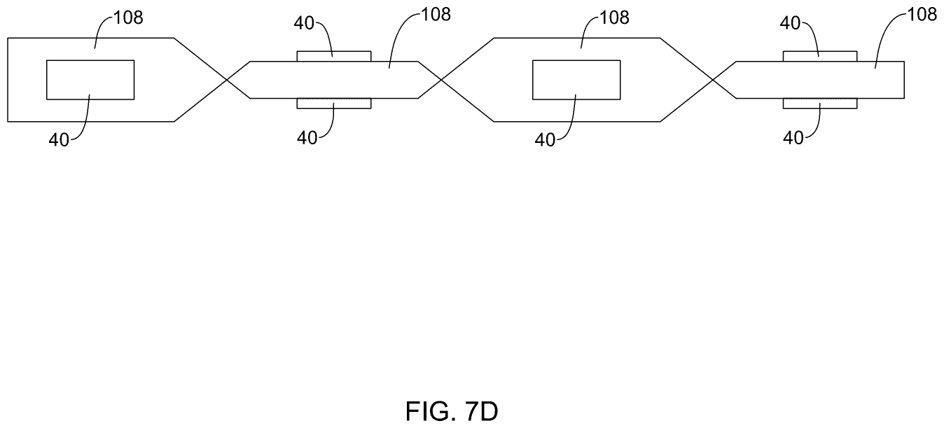

FIG. 7A illustrates one example technique for arranging the components of the ultrasound assembly 42 (as schematically illustrated in FIG. 5) into the inner core 34 (as schematically illustrated in FIG. 4). FIG. 7A is a cross-sectional view of the ultrasound assembly 42 taken within group G1 in FIG. 5, as indicated by the presence of four lead wires 110. For example, if a cross-sectional view of the ultrasound assembly 42 was taken within group G4 in FIG. 5, only one lead wire 110 would be present (that is, the one lead wire connecting group G5).

Referring still to FIG. 7A, the common wire 108 includes an elongate, flat piece of electrically conductive material in electrical contact with a pair of ultrasound radiating members 40. Each of the ultrasound radiating members 40 is also in electrical contact with a positive contact wire 112. Because the common wire 108 is connected to the negative terminal 106, and the positive contact wire 112 is connected to the positive terminal 104, a voltage difference can be created across each ultrasound radiating member 40. Lead wires 110 may be separated from the other components of the ultrasound assembly 42, thus preventing interference with the operation of the ultrasound radiating members 40 as described above. For example, in one embodiment, the inner core 34 may be filled with an insulating potting material 43, thus deterring unwanted electrical contact between the various components of the ultrasound assembly 42.

FIGS. 7B-7C illustrate cross sectional views of the inner core 34 of FIG. 7A taken along lines 7B-7B and 7C-7C, respectively. As illustrated in FIG. 7B, the ultrasound radiating members 40 are mounted in pairs along the common wire 108. The ultrasound radiating members 40 are connected by positive contact wires 112, such that substantially the same voltage is applied to each ultrasound radiating member 40. As illustrated in FIG. 7C, the common wire 108 may include wide regions 108W upon which the ultrasound radiating members 40 can be mounted, thus reducing the likelihood that the paired ultrasound radiating members 40 will short together. In certain embodiments, outside the wide regions 108W, the common wire 108 may have a more conventional, rounded wire shape.

In an embodiment, such as illustrated in FIG. 7D, the common wire 108 may be twisted to form a helical shape before being fixed within the inner core 34. In such embodiments, the ultrasound radiating members 40 are oriented in a plurality of radial directions, thus enhancing the radial uniformity of the resulting ultrasonic energy field.

One of ordinary skill in the art will recognize that the wiring arrangement described above can be modified to allow each group G1, G2, G3, G4, G5 to be independently powered. Specifically, by providing a separate power source within the control system 100 for each group, each group can be individually turned on or off, or can be driven with an individualized power. This provides the advantage of allowing the delivery of ultrasonic energy to be “turned off” in regions of the treatment site where treatment is complete, thus preventing deleterious or unnecessary ultrasonic energy to be applied to the patient.

The embodiments described above, and illustrated in FIGS. 5-7, illustrate a plurality of ultrasound radiating members grouped spatially. That is, in such embodiments, all of the ultrasound radiating members within a certain group are positioned adjacent to each other, such that when a single group is activated, ultrasonic energy is delivered at a specific length of the ultrasound assembly. However, in some embodiments, the ultrasound radiating members of a certain group may be spaced apart from each other, such that the ultrasound radiating members within a certain group are not positioned adjacent to each other. In such embodiments, when a single group is activated, ultrasonic energy can be delivered from a larger, spaced apart portion of the energy delivery section. Such modified embodiments may be advantageous in applications wherein it is desired to deliver a less focused, more diffuse ultrasonic energy field to the treatment site.

In some embodiments, the ultrasound radiating members 40 may include rectangular lead zirconate titanate (“PZT”) ultrasound transducers that have dimensions of about 0.017 inches (about 0.04318 centimeters) by about 0.010 inches (about 0.0254 centimeters) by about 0.080 inches (about 0.2032 centimeters). In other embodiments, other configurations may be used. For example, disc-shaped ultrasound radiating members 40 can be used in other embodiments. In an embodiment, the common wire 108 includes copper, and is about 0.005 inches (about 0.0127 centimeters) thick, although other electrically conductive materials and other dimensions can be used in other embodiments. Lead wires 110 may be 36 gauge electrical conductors, for example, while positive contact wires 112 may be 42 gauge electrical conductors. However, one of ordinary skill in the art will recognize that other wire gauges can be used in other embodiments.

As described above, suitable frequencies for the ultrasound radiating member 40 include, but are not limited to, from about 20 kHz to about 20 MHz. In one embodiment, the frequency is between about 500 kHz and 20 MHz, and in another embodiment 1 MHz and 3 MHz. In yet another embodiment, the ultrasound radiating members 40 are operated with a frequency of about 2 MHz.

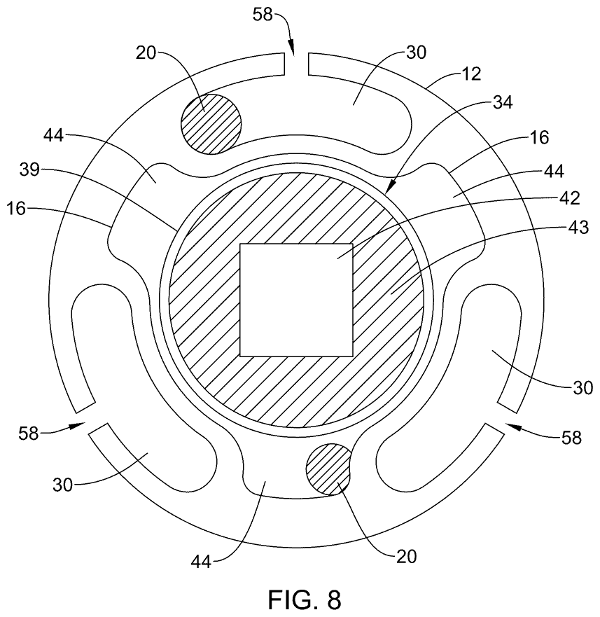

FIG. 8 illustrates the inner core 34 positioned within the catheter shaft 12. Details of the ultrasound assembly 42, provided in FIG. 7A, are omitted for clarity. As described above, the inner core 34 can be slid within the central lumen 51 of the catheter shaft 12, thereby allowing the inner core energy delivery section 41 (e.g., see FIG. 3) to be positioned within the tubular body energy delivery section 18. For example, in an embodiment, the materials including the inner core energy delivery section 41, the tubular body energy delivery section 18, and the potting material 43 may all be materials having a similar acoustic impedance, thereby minimizing ultrasonic energy losses across material interfaces.

FIG. 8 further illustrates placement of fluid delivery ports 58 within the tubular body energy delivery section 18. As illustrated, holes or slits are formed from the fluid delivery lumen 30 through the catheter shaft 12, thereby permitting fluid flow from the fluid delivery lumen 30 to the treatment site. Thus, a source of therapeutic compound coupled to the inlet port 32 provides a hydraulic pressure which drives the therapeutic compound through the fluid delivery lumens 30 and out the fluid delivery ports 58.

By evenly spacing the fluid delivery lumens 30 around the circumference of the catheter shaft 12, as illustrated in FIG. 8, a substantially even flow of therapeutic compound around the circumference of the catheter shaft 12 can be achieved. In addition, the size, location and geometry of the fluid delivery ports 58 can be selected to provide uniform fluid flow from the fluid delivery ports 30 to the treatment site. For example, in one embodiment, fluid delivery ports 58 closer to the proximal region of the energy delivery section 18 have smaller diameters then fluid delivery ports 58 closer to the distal region of the energy delivery section 18, thereby allowing uniform delivery of fluid across the entire energy delivery section.

For example, in one embodiment in which the fluid delivery ports 58 have similar sizes along the length of the catheter shaft 12, the fluid delivery ports 58 have a diameter between about 0.0005 inches (about 0.00127 centimeters) to about 0.0050 inches (about 0.0127 centimeters). In another embodiment in which the size of the fluid delivery ports 58 changes along the length of the catheter shaft 12, the fluid delivery ports 58 have a diameter between about 0.001 inches (about 0.00254 centimeters) to about 0.005 inches (about 0.0127 centimeters) in the proximal region of the energy delivery section 18 (e.g., see FIG. 1), and between about 0.005 inches (about 0.0127 centimeters) to 0.020 inches (0.0508 centimeters) in the distal region of the energy delivery section 18. The increase in size between adjacent fluid delivery ports 58 depends on the material comprising the catheter shaft 12, and on the size of the fluid delivery lumen 30. The fluid delivery ports 58 can be created in the catheter shaft 12 by punching, drilling, burning or ablating (such as with a laser), or by any other suitable method. Therapeutic compound flow along the length of the catheter shaft 12 can also be increased by increasing the density of the fluid delivery ports 58 toward the distal end region 15 of the catheter shaft 12.

In the case of delivery of cavitation nuclei such as microbubbles, nanobubbles, microdroplets or nanodroplets, it can be beneficial to make the fluid delivery ports 58 large enough so that the cavitation nuclei are not subject to excessive pressure or shear stresses as the cavitation nuclei traverse the fluid delivery lumens 30 and exit the fluid delivery ports 58. It should be appreciated that it may be desirable to provide non-uniform fluid flow from the fluid delivery ports 58 to the treatment site. In such embodiments, the size, location and geometry of the fluid delivery ports 58 can be selected to provide such non-uniform fluid flow.

Referring still to FIG. 8, placement of the inner core 34 within the catheter shaft 12 further defines cooling fluid lumens 44. Cooling fluid lumens 44 are formed between an outer surface 39 of the inner core 34 and an inner surface 16 of the catheter shaft 12. In some embodiments, a cooling fluid may be introduced through the proximal access port 31 such that cooling fluid flow is produced through cooling fluid lumens 44 and out the distal exit port 29 (see FIG. 1). In some cases, the cooling fluid lumens 44 may be evenly spaced around the circumference of the catheter shaft 12 (that is, at about 120° increments for a three-lumen configuration), thereby providing uniform cooling fluid flow over the inner core 34. Such a configuration is useful for removing unwanted thermal energy at the treatment site. The flow rate of the cooling fluid and the power to the ultrasound assembly 42 may be adjusted to maintain the temperature of the distal end region 15 of the catheter 10 within a desired range. In some cases, the desired temperature range may be between 28° C. and 52° C. In some cases, the desired temperature range may be between 28° C. and 45° C. In some cases, the desired temperature range may be between 28° C. and 43° C.

In an embodiment, the inner core 34 can be rotated or moved within the catheter shaft 12. Specifically, movement of the inner core 34 can be accomplished by maneuvering the proximal hub 37 while holding the hub 33 stationary. The inner core outer body 35 is at least partially constructed from a material that provides enough structural support to permit movement of the inner core 34 within the catheter shaft 12 without kinking of the catheter shaft 12. Additionally, the inner core outer body 35 may include a material having the ability to transmit torque. Suitable materials for the inner core outer body 35 include, but are not limited to, polyimides, polyesters, polyurethanes, thermoplastic elastomers and braided polyimides.

In an embodiment, the fluid delivery lumens 30 and the cooling fluid lumens 44 are open at the distal end of the catheter shaft 12, thereby allowing the therapeutic compound and the cooling fluid to pass into the patient's vasculature at the distal exit port. Or, if desired, the fluid delivery lumens 30 can be selectively occluded at the distal end of the catheter shaft 12, thereby providing additional hydraulic pressure to drive the therapeutic compound out of the fluid delivery ports 58. In either configuration, the inner core 34 can be prevented from passing through the distal exit port by making the inner core 34 with a length that is less than the length of the tubular body. In other embodiments, a protrusion is formed on the internal side of the tubular body in the distal end region 15, thereby preventing the inner core 34 from passing through the distal exit port.

In still other embodiments, the catheter 10 may further include an occlusion device (not shown) positioned at the distal exit port 29. The occlusion device may have a reduced inner diameter that can accommodate a guidewire, but that is less than the inner diameter of the central lumen 51. Thus, the inner core 34 is prevented from extending through the occlusion device and out the distal exit port 29. For example, suitable inner diameters for the occlusion device include, but are not limited to, about 0.005 inches (about 0.0127 centimeters) to about 0.050 inches (about 0.127 centimeters). In other embodiments, the occlusion device has a closed end, thus preventing cooling fluid from leaving the catheter 10, and instead recirculating to the proximal end region 14 of the catheter shaft 12. These and other cooling fluid flow configurations permit the power provided to the ultrasound assembly 42 to be increased in proportion to the cooling fluid flow rate. Additionally, certain cooling fluid flow configurations can reduce exposure of the patient's body to cooling fluids.

In certain embodiments, as illustrated in FIG. 8, the catheter shaft 12 may further include one or more temperature sensors 20, that may be located within the energy delivery section 18. In such embodiments, the proximal end region 14 of the catheter shaft 12 includes a temperature sensor lead which can be incorporated into cable 45 (illustrated in FIG. 1). Suitable temperature sensors include, but are not limited to, temperature sensing diodes, thermistors, thermocouples, resistance temperature detectors (“RTDs”) and fiber optic temperature sensors which use thermalchromic liquid crystals. Suitable temperature sensor 20 geometries include, but are not limited to, a point, a patch or a stripe. The temperature sensors 20 can be positioned within one or more of the fluid delivery lumens 30 (as illustrated), and/or within one or more of the cooling fluid lumens 44.

The ultrasound radiating members may be operated in a pulsed mode. For example, in one embodiment, the time average electrical power supplied to the ultrasound radiating members 40 is between about 0.001 watts and about 5 watts and can be between about 0.05 watts and about 3 watts. In some embodiments, the time average electrical power over treatment time is about 0.45 watts or 1.2 watts. The duty cycle is between about 0.01% and about 90% and can be between about 0.1% and about 50%. In certain embodiments, the duty ratio is about 7.5%, 15% or a variation between 1% and 30%. The pulse averaged electrical power for each ultrasound radiating member 40 can be between about 0.01 watts and about 20 watts and can be between about 0.1 watts and 20 watts. In certain embodiments, the pulse averaged electrical power is about 4 watts, 8 watts, 16 watts, or a variation of 0.5 to 8 watts. As described above, the amplitude, pulse width, pulse repetition frequency, peak negative acoustic pressure or any combination of these parameters can be constant or varied during each pulse or over a set of pulses. In a non-linear application of acoustic parameters the above ranges can change significantly. Accordingly, the overall time average electrical power over treatment time may stay the same but not real-time average power.

In one embodiment, the pulse repetition rate may be between about 1 Hz and about 2 kHz and more can be between about 1 Hz and about 50 Hz. In another embodiment, the pulse repetition rate is about 30 Hz, or a variation of about 10 Hz to about 40 Hz. The pulse duration or width can be between about 0.5 millisecond and about 50 milliseconds and can be between about 0.1 millisecond and about 25 milliseconds. In some embodiments, the pulse duration is about 2.5 milliseconds, 5 or a variation of 1 to 8 milliseconds. In addition, the peak negative acoustic pressure can be between about 0.1 to about 50 MPa or in another embodiment between about 0.5 to about 2.0 MPa.

In one embodiment, the transducers are operated at an average power of about 0.6 watts, a duty cycle of about 7.5%, a pulse repetition rate of about 30 Hz, a pulse average electrical power of about 8 watts and a pulse duration of about 2.5 milliseconds.

The ultrasound radiating member used with the electrical parameters described herein may have an acoustic efficiency greater than about 50% and can be greater than about 75%. The ultrasound radiating member can be formed a variety of shapes, such as, cylindrical (solid or hollow), flat, bar, triangular, and the like. The length of the ultrasound radiating member may be between about 0.1 centimeters (cm) and about 0.7 cm. The thickness or diameter of the ultrasound radiating members may be between about 0.02 cm and about 0.5 cm.

In certain embodiments, the therapeutic compound delivered to the treatment site includes a plurality of bubbles, for example microbubbles, having, for example, a gas formed therein. Exemplary gases that are usable to form the microbubbles include, but are not limited to, air, oxygen, carbon dioxide, perfluorocarbon gases and inert gases.

In some embodiments, the microbubble-therapeutic compound can include about 104 microbubbles per milliliter of liquid to about 1010 microbubbles per millimeter of liquid, or from about 106 to about 109 microbubbles per milliliter of liquid. In some embodiments the microbubbles in the microbubble-therapeutic compound have a diameter of between about 0.1 micrometers (μm) and about 30 μm. In some embodiments, the microbubbles have a diameter of about 0.1 to about 10 μm, about 0.2 to about 10 μm, about 0.5 to about 10 μm, about 0.5 to about 5 μm, or about 1 μm. In some embodiments, the microbubbles have a diameter of less than or equal to about 10 μm, about 5 μm, or about 2.5 μm. Other parameters can be used in other embodiments.

In some embodiments, the efficacy of the therapeutic compound is enhanced by the presence of the microbubbles contained therein. In some embodiments, the microbubbles can act as a nucleus for cavitation, and thus allow cavitation to be induced at lower levels of peak rarefaction acoustic pressure. Therefore, a reduced amount of peak rarefaction acoustic pressure can be delivered to the treatment site without reducing the efficacy of the treatment. Reducing the amount of ultrasonic pressure delivered to the treatment site reduces risks associated with overheating the treatment site, and, in certain embodiments, also reduces the time required to treat a vessel. In some embodiments, cavitation also promotes more effective diffusion and penetration of the therapeutic compound into surrounding tissues, such as the vessel wall and/or the clot material. Furthermore, in some embodiments, the mechanical agitation caused by cavitation of the microbubbles is effective in mechanically breaking up clot material.

In can be appreciated that the interaction between a fluid (e.g., a therapeutic material) and/or microbubbles delivered through fluid delivery lumens with ultrasound energy transmitted by the ultrasound transmitters may cause the microbubbles to be disrupted and/or otherwise burst. The bursting of the microbubbles at or adjacent the target site may help to increase the effectiveness/efficacy of the therapeutic material. It can also be appreciated that if the microbubbles are burst before the therapeutic material reaches the target site, the benefits of the microbubbles may be reduced or lost. In other words, premature bursting of the microbubbles may reduce the effectiveness of the fluid/therapeutic material and/or the treatment in general. Disclosed herein are systems that designed to reduce the likelihood that microbubbles delivered through fluid delivery lumens are prematurely burst and/or otherwise maximize the therapeutic benefit of the microbubbles.

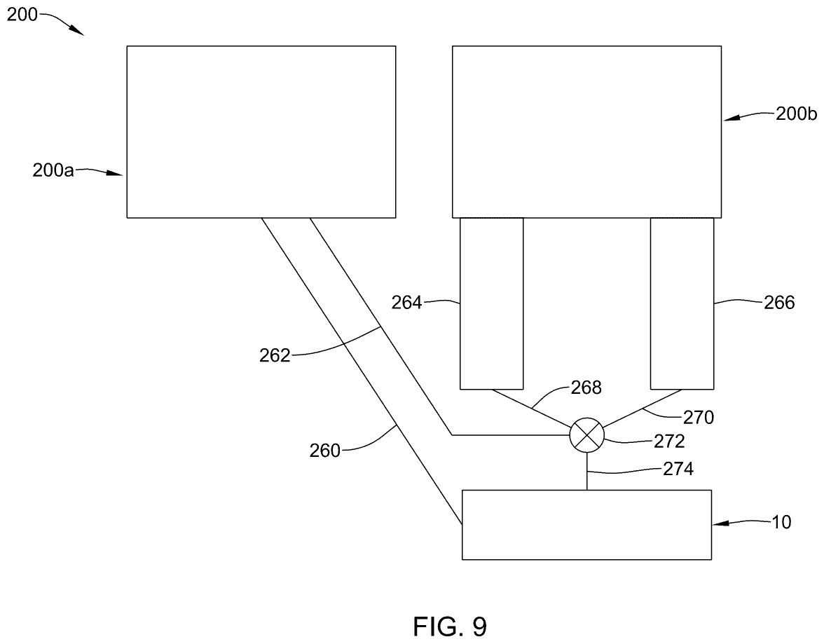

FIG. 9 schematically depicts an example system 200. In general, the system 200 is configured to reduce the likelihood that microbubbles delivered through fluid delivery lumens are prematurely burst and/or otherwise maximize the therapeutic benefit of the microbubbles. The system 200 may include the ultrasonic catheter 10 (e.g., as disclosed herein, may also be termed the catheter 10, the catheter shaft 10, etc.) or a similar ultrasonic catheter or catheter shaft. While not shown in FIG. 9, the catheter shaft 10 may include structural features disclosed herein including a treatment core 34, one or more ultrasound transducers 40 coupled to the treatment core 34, one or more fluid delivery lumens 30, etc. In some of these and in other instances, the ultrasonic catheter 10 may be the treatment core 34. A control unit 200a and a pump 200b may be coupled to the catheter shaft 10. For example, the control unit 200a may be coupled to the catheter shaft 10 along a pathway 260. In at least some instances, the control unit 200a may be coupled to the catheter shaft 10 by attaching the catheter shaft 10 (e.g., the treatment core 34) directly to the to the control unit 200a. Alternatively, one or more connectors, conductors, and/or the like may extend between the catheter shaft 10 and the control unit 200a (e.g., along the pathway 260) so that, for example, the control unit 200a can be used to control the treatment core 34 and/or the ultrasound transducers 40.

In some instances, the pump 200b may be configured to deliver one or more fluids (e.g., saline, therapeutic material, microbubbles, nanodroplets, combinations thereof, and/or the like) to the catheter 10 (e.g., the fluid delivery lumens 30). For example, the pump 200b may be used to deliver fluid from a first fluid source 264 and/or a second fluid source 266. The first fluid source 264 may include a therapeutic material, a thrombolytic material, microbubbles, nanodroplets, combinations thereof, and/or the like. The second fluid source 266 may include saline, a biocompatible fluid, a solution, a mixture, a fluid, and/or the like. Other materials are contemplated. A switch 272 may be used to switch between delivering fluid from the first fluid source 264 and delivering fluid from the second fluid source 266. For example, the switch 272 may be used to switch between delivering (a) a thrombolytic material in combination with microbubbles, nanodroplets, and/or the like, and (b) saline (and/or a biocompatible fluid, a solution, a mixture, a fluid, and/or the like). The first fluid source 264 may be coupled to the switch 272 along a pathway 268. The second fluid source 266 may be coupled to the switch 272 along a pathway 270. The switch 272 may be coupled to the catheter 10 along a pathway 274. In some instances, the control unit 200a may also be coupled to the switch 272 along a pathway 262.

As indicated above, it may be desirable to reduce the likelihood that microbubbles delivered through fluid delivery lumens are prematurely burst and/or otherwise maximize the therapeutic benefit of the microbubbles. The system 200 is configured so that the control unit 200a and the pump 200b can be coordinated (e.g., via a control algorithm) so that the therapeutic material and microbubbles are delivered through the fluid delivery lumens while the ultrasound transducers are not active. For example, the control unit 200a may be configured to shift between in a first configuration or state where the therapeutic core 34 and/or the plurality of ultrasound transducers 40 are in an active state (e.g., active, energized, etc.) and a second configuration or state where the therapeutic core 34 and/or the plurality of ultrasound transducers 40 are in an inactive state (e.g., inactive, not energized, etc.). The pump 200b may be configured to deliver microbubbles, nanodroplets, or both (e.g., along with the therapeutic material) to the fluid delivery lumens 30 when the control unit 200a is in the second configuration/state (e.g., when the ultrasound transducers 40 are inactive). The pump 200b may also be configured to not deliver microbubbles, nanodroplets, or both (e.g., along with the therapeutic material) to the fluid delivery lumens 30 when the control unit 200a is in the first configuration/state (e.g., when the ultrasound transducers 40 are active). In at least some instances, the pump 200b may deliver saline to the fluid delivery lumens 30 when the control unit 200a is in the first configuration/state (e.g., when the ultrasound transducers 40 are active). In some of these and in other instances, the pump 200b may be configured to deliver microbubbles, nanodroplets, or both to the fluid delivery lumen for a period of time that can be controlled and can vary from being in phase with the period of time the ultrasound transducers 40 are active to being out of phase with the period of time the ultrasound transducers 40 are active. The pump 200b may be configured to deliver saline, a biocompatible fluid, a solution, a mixture, a fluid, and/or the like through the fluid delivery lumen 30 when not delivering microbubbles, nanodroplets, or both to the fluid delivery lumen 30.

The switch 272 may be configured to switch between a first switch configuration where the pump 200b delivers microbubbles, nanodroplets, or both along with a therapeutic material (e.g. from the first fluid source 264) to the fluid delivery lumens 30 and a second switch configuration where the pump 200b delivers saline, a biocompatible fluid, a solution, a mixture, a fluid, and/or the like (e.g., from the second fluid source 266) to the fluid delivery lumens 30. In some instances, the control unit 200a may be coupled to the switch 272 so as to control or otherwise activate the switch 272. This may allow the control unit 200a to control whether the pump 200b delivers microbubbles, nanodroplets, or both along with a therapeutic material (e.g. from the first fluid source 264) or saline, a biocompatible fluid, a solution, a mixture, a fluid, and/or the like (e.g., from the second fluid source 266) to the fluid delivery lumens 30.

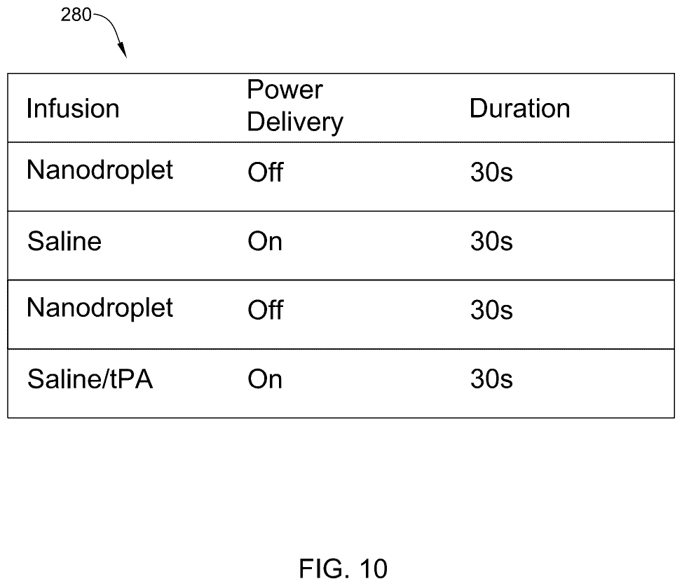

FIG. 10 schematically depicts a control algorithm for use with the system 200 (and/or other systems disclosed herein). In this example, power delivery may be shifted between “on” and “off”. In this example, power delivery being “on” may correspond to the control unit 200a being in the first state where the therapeutic core 34 and/or the plurality of ultrasound transducers 40 are in an active state. Power delivery being “off” may correspond to the control unit 200a being in the second state where the therapeutic core 34 and/or the plurality of ultrasound transducers 40 are in an inactive state. When the power delivery is “off” (e.g., the control unit 200a is in the second state), the pump 200b may be used to deliver/infuse a thrombolytic material in combination with microbubbles, nanodroplets, and/or the like. When the power delivery is “on” (e.g., the control unit 200a is in the first state), the pump 200b may be used to deliver/infuse saline, a biocompatible fluid, a solution, a mixture, a fluid, and/or the like. The control unit 200a may activate the switch 272 to the appropriate fluid source 264, 266 depending on the state/configuration of the control unit 200a. In some instances, the control unit 200a may remain in either the first state or the second state or otherwise activate/inactivate the ultrasound transducers 40 for a duration of time. For example, when the control unit is in the first state, the plurality of ultrasound transducers 40 may be in the active state or otherwise active/energized for about 10-60 seconds, or about 15-45 seconds, or about 30 seconds. When the control unit is in the second state, the plurality of ultrasound transducers 40 may be in the inactive state or otherwise inactive for about 10-60 seconds, or about 15-45 seconds, or about 30 seconds.

The materials that can be used for the various components of the devices described herein may include those commonly associated with medical devices. The devices and components thereof described herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the devices described herein to achieve the same result.

In some embodiments, a degree of Magnetic Resonance Imaging (MRI) compatibility is imparted into the devices described herein. For example, the devices described herein, or portions thereof, may be made of a material that does not substantially distort the image and create substantial artifacts (e.g., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system for treating a vascular region, the system comprising:
an elongate catheter shaft having a distal end region;
wherein a central lumen is formed in the elongate catheter shaft;
a treatment core disposed within the central lumen, the treatment core including a plurality of ultrasound transducers disposed adjacent to the distal end region of the elongate catheter shaft;
a control unit coupled to the treatment core, the control unit being configured to shift between in a first configuration where the plurality of ultrasound transducers are in an active state and a second configuration where the plurality of ultrasound transducers are in an inactive state;
a fluid delivery lumen defined in the elongate catheter shaft; and
a pump coupled to the elongate catheter shaft, the pump being configured to deliver microbubbles, nanodroplets, or both to the fluid delivery lumen when the ultrasound transducers are in the inactive state.

2. The system of claim 1, wherein the pump is configured to deliver saline through the fluid delivery lumen when the when the ultrasound transducers are in the active state.

3. The system of claim 1, wherein the pump includes a switch.

4. The system of claim 3, wherein the switch is configured to switch between a first switch configuration where the pump delivers microbubbles, nanodroplets, or both to the fluid delivery lumen and a second switch configuration where the pump delivers saline to the fluid delivery lumen.

5. The system of claim 1, wherein when the control unit is in the first configuration, the plurality of ultrasound transducers are in the active state for about 10-60 seconds.

6. The system of claim 1, wherein when the control unit is in the first configuration, the plurality of ultrasound transducers are in the active state for about 15-45 seconds.

7. The system of claim 1, wherein when the control unit is in the first configuration, the plurality of ultrasound transducers are in the active state for about 30 seconds.

8. The system of claim 1, wherein when the control unit is in the second configuration, the plurality of ultrasound transducers are in the inactive state for about 10-60 seconds.

9. The system of claim 1, wherein when the control unit is in the second configuration, the plurality of ultrasound transducers are in the inactive state for about 15-45 seconds.

10. The system of claim 1, wherein when the control unit is in the second configuration, the plurality of ultrasound transducers are in the inactive state for about 30 seconds.

11. The system of claim 1, wherein the pump is configured to not deliver microbubbles, nanodroplets, or both to the fluid delivery lumen when the ultrasound transducers are in the active state.

12. A system for treating a vascular region, the system comprising:
an elongate catheter shaft;
a treatment core disposed within the elongate catheter shaft, the treatment core including a plurality of ultrasound transducers;
a control unit coupled to the treatment core, the control unit being configured to shift between in a first state where the plurality of ultrasound transducers are active and a second state where the plurality of ultrasound transducers are inactive;
a fluid delivery lumen defined in the elongate catheter shaft; and
a pump coupled to the elongate catheter shaft, wherein the pump is configured to deliver microbubbles, nanodroplets, or both to the fluid delivery lumen when the control unit is in the first state and wherein the pump is configured to not deliver microbubbles, nanodroplets, or both to the fluid delivery lumen when the control unit is in the second state.

13. The system of claim 12, wherein the pump is configured to deliver saline through the fluid delivery lumen when the control unit is in the second state.

14. The system of claim 12, wherein the pump includes a switch.

15. The system of claim 14, wherein the switch is configured to switch between a first switch configuration when the control unit is in the first state so that the pump delivers microbubbles, nanodroplets, or both to the fluid delivery lumen and a second switch configuration when the control unit is in the second state so that the pump delivers saline to the fluid delivery lumen.

16. The system of claim 12, wherein when the control unit is in the first state, the plurality of ultrasound transducers are active for about 10-60 seconds.

17. The system of claim 12, wherein when the control unit is in the first state, the plurality of ultrasound transducers are active for about 15-45 seconds.

18. The system of claim 12, wherein when the control unit is in the first state, the plurality of ultrasound transducers are active for about 30 seconds.

19. The system of claim 12, wherein when the control unit is in the second state, the plurality of ultrasound transducers are inactive for about 10-60 seconds.

20. A method for delivering a drug to a vascular region, the method comprising:
advancing a catheter system to a treatment site, the catheter system comprising:
an elongate catheter shaft having a distal end region,
wherein a central lumen is formed in the elongate catheter shaft,
a treatment core disposed within the central lumen, the treatment core including a plurality of ultrasound transducers disposed adjacent to the distal end region of the elongate catheter shaft,
a control unit coupled to the treatment core, the control unit being configured to shift between in a first configuration where the plurality of ultrasound transducers are in an active state and a second configuration where the plurality of ultrasound transducers are in an inactive state,
a fluid delivery lumen defined in the elongate catheter shaft, and
a pump coupled to the elongate catheter shaft, the pump being configured to deliver microbubbles, nanodroplets, or both to the fluid delivery lumen when the control unit is in the inactive state;
advancing the treatment core through the central lumen so that the plurality of ultrasound transducers are disposed adjacent to the distal end region of the elongate catheter shaft;
delivering microbubbles, nanodroplets, or both to the fluid delivery lumen when the control unit is in the inactive state; and
activating at least some of the plurality of ultrasound transducers.
